(19) United States
(12) Patent Application Publication
(10) Pub. No.: US 2022/0054329 A1
(43) Pub. Date: Feb. 24, 2022

(54) MOBILE PHYSICAL THERAPY SYSTEM, APPARATUS AND METHOD

(71) Applicants: Ryan Edward Elassal, Edmond, OK (US); Michael Steven Hayes, Oklahoma City, OK (US)

(72) Inventors: Ryan Edward Elassal, Edmond, OK (US); Michael Steven Hayes, Oklahoma City, OK (US)

(21) Appl. No.: 16/999,608

(22) Filed: Aug. 21, 2020

(51) Int. Cl. A61G 3/00; A61N 1/36; A61N 7/00; A63B 21/062; A63B 22/02; A61G 13/00

(52) U.S. Cl. CPC A61G 3/001; A61N 1/36014; A61N 7/00; A63B 2022/0652; A63B 22/02; A61G 13/009; A63B 21/062

(57) ABSTRACT

A mobile physical therapy apparatus to be transported by a mobile physical therapy vehicle. The mobile physical therapy apparatus includes a housing detachably connected to an interior of the mobile physical therapy vehicle; a treadmill positioned within and detachably connected to the housing of the mobile physical therapy vehicle; a recumbent bicycle positioned within and detachably connected to the housing of the mobile physical therapy vehicle; a pulley tower weight training system positioned within and detachably connected to the housing of the mobile physical therapy vehicle; an electrical stimulation and ultrasound apparatus positioned within and detachably connected to the housing of the mobile physical therapy vehicle; and a movable patient support table positioned within and detachably connected to the housing of the mobile physical therapy vehicle.

### FIELD OF INVENTION

The invention is in the technical field of mobile physical therapy systems, apparatuses and methods as described and claimed herein. The invention is generally related to vehicles with specialized mobile physical therapy equipment detachably connected thereto. The vehicle is configured to permit a licensed physical therapist to provide prescribed physical therapy to patients at remote locations. Thus, reducing or eliminating the need for patients to travel to designated healthcare facilities to receive physical therapy.

### BACKGROUND

Physical therapy is a specialized type of healthcare that is focused on easing pain and helping a patient function, move and live a better more productive life. Traditionally, physical therapy is prescribed by a licensed healthcare provider, such as a doctor, as a treatment for certain physical ailments and is administered by a licensed physical therapist. A licensed physical therapist may provide, for example, a physical examination and evaluation of the patient, including a review of health history and certain testing procedures, including evaluating posture, movement, flexibility and muscle and joint motion and performance. A physical therapist may also provide a diagnosis, plan of care and short and long-term goals for improving a patient's physical abilities. The physical therapy prescribed may include specially designed exercises and use of specially designed equipment located at a healthcare facility.

Unfortunately, however, while such healthcare facilities may be convenient for providers of physical therapy, such facilities are often inconvenient for patients with physical ailments who must travel to and from such facilities to receive treatment. Often, a patient will elect not to do physical therapy, simply to avoid the inconvenience and pain associated with travel to and from such facilities. Further, because traditional physical therapy equipment is only used for a specified treatment period, is expensive, heavy and specialized, it is not practical, cost effective, or efficient for patients to purchase such equipment for use at their home. Thus, making traditional physical therapy equipment systems, apparatuses and methods inefficient, inconvenient and not practical for remote use or in-home use. Thereby decreasing access to necessary healthcare and decreasing the overall health and general well-being of patients in need of physical therapy.

To improve the health of people in need of physical therapy, therefore, it would be advantageous to provide an improved mobile physical therapy system, apparatus and method as described and claimed herein.

### SUMMARY OF THE INVENTION

The inventive concepts disclosed herein are generally directed to an improved mobile physical therapy system, apparatus and method as described and claimed herein. In some embodiments, the inventive concepts disclosed herein are directed to a mobile physical therapy apparatus to be transported by a mobile physical therapy vehicle. In some embodiments, the mobile physical therapy apparatus includes a housing detachably connected to an interior of the mobile physical therapy vehicle; a treadmill positioned within and detachably connected to the housing of the mobile physical therapy vehicle; a recumbent bicycle positioned within and detachably connected to the housing of the mobile physical therapy vehicle; a pulley tower weight training system positioned within and detachably connected to the housing of the mobile physical therapy vehicle; an electrical stimulation and ultrasound apparatus positioned within and detachably connected to the housing of the mobile physical therapy vehicle; and a movable patient support table positioned within and detachably connected to the housing of the mobile physical therapy vehicle. In some embodiments, the physical therapy equipment described herein is configured to be selectively removable and selectively detachable such that each piece of equipment may be selectively added, removed and rearranged within the housing of the mobile physical therapy vehicle depending upon the needs of the patient. Further, in some embodiments, the physical therapy equipment is configured to be selectively movable or slidable within the housing of the mobile physical therapy vehicle, from a stowed position (when not in use), to a deployed position for use by a patient. In this way, the mobile physical therapy equipment may be customizable, based on patient needs, for any type of physical therapy.

Further, in some embodiments, the inventive concepts disclosed herein include a method for delivering mobile physical therapy. The method including the steps of: receiving a prescription to provide physical therapy to a patient; confirming that the prescription was prescribed by an authorized health care professional; scheduling an appointment with the patient to provide mobile physical therapy to the patient; and providing physical therapy to the patient in a mobile physical therapy vehicle.

Further, in some embodiments, the inventive concepts disclosed herein include a system for providing mobile physical therapy. The system including a mobile physical therapy vehicle. The mobile physical therapy vehicle including a treadmill positioned within and detachably connected to an interior portion of the mobile physical therapy vehicle; a recumbent bicycle positioned within and detachably connected to an interior portion of the mobile physical therapy vehicle; a pulley tower weight training system positioned within and detachably connected to an interior portion of the mobile physical therapy vehicle; an electrical stimulation and ultrasound apparatus positioned within and detachably connected to an interior portion of the mobile physical therapy vehicle; and a movable patient support table positioned within and detachably connected to an interior portion of the mobile physical therapy vehicle. In some embodiments, the physical therapy equipment that is part of the system for providing mobile physical therapy is configured to be selectively removable and selectively detachable such that each piece of equipment may be selectively added, removed and rearranged within the mobile physical therapy vehicle depending upon the needs of the patient. Further, in some embodiments, the physical therapy equipment is configured to be selectively movable or slidable within the mobile physical therapy vehicle, from a stowed position (when not in use), to a deployed position for use by a patient. In this way, the mobile physical therapy equipment may be customizable, based on patient needs, for any type of physical therapy.

In some embodiments, the physical therapy equipment described herein, including specifically, the pulley tower weight training system is configured to be locked or fixed in place and secured to the housing or the mobile physical therapy vehicle for added stability and safety during use. The physical therapy equipment, including the pulley tower weight training system may be locked in place using bolts, screws, pins, welded joints, straps and locks, combinations thereof and the like for example.

It is to such an improved mobile physical therapy system, apparatus and to methods of using thereof that exemplary embodiments of the inventive concepts disclosed and claimed herein are directed.

### BRIEF DESCRIPTION OF THE SEVERAL VIEWS OF THE DRAWINGS

Like reference numerals in the figures represent and refer to the same or similar element or function. Implementations of the disclosure may be better understood when consideration is given to the following detailed description thereof. Such description makes reference to the annexed pictorial illustrations, schematics, graphs, drawings, and appendices. In the drawings:

FIG. 1 is a side perspective view of a mobile physical therapy vehicle (105) according to the inventive concepts disclosed herein.

FIG. 2 is a mobile physical therapy apparatus (100) according to the inventive concepts disclosed herein.

FIG. 3 is a diagram of a method (200) for delivering mobile physical therapy according to the inventive concepts disclosed herein.

FIG. 4 is a side perspective view of a system (300) for providing mobile physical therapy according to the inventive concepts disclosed herein.

### DETAILED DESCRIPTION OF EXEMPLARY EMBODIMENTS

Before explaining at least one embodiment of the inventive concepts disclosed herein in detail, it is to be understood that the inventive concepts are not limited in their application to the details of construction and the arrangements of the components or steps or methodologies set forth in the following description or illustrated in the drawings. The inventive concepts disclosed herein are capable of other embodiments or of being practiced or carried out in various ways. Also, it is to be understood that the phraseology and terminology employed herein is for the purpose of description and should not be regarded as limiting the inventive concepts claimed herein in any way.

In the following detailed description of embodiments of the inventive concepts, numerous specific details are set forth in order to provide a more thorough understanding of the inventive concepts. However, it will be apparent to one of ordinary skill in the art that the inventive concepts within the disclosure may be practiced without these specific details, in other instances, well-known features have not been described in detail to avoid unnecessarily complicating the instant disclosure,

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

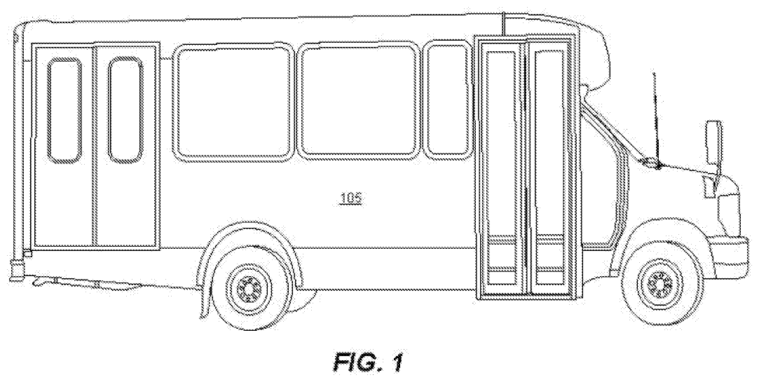

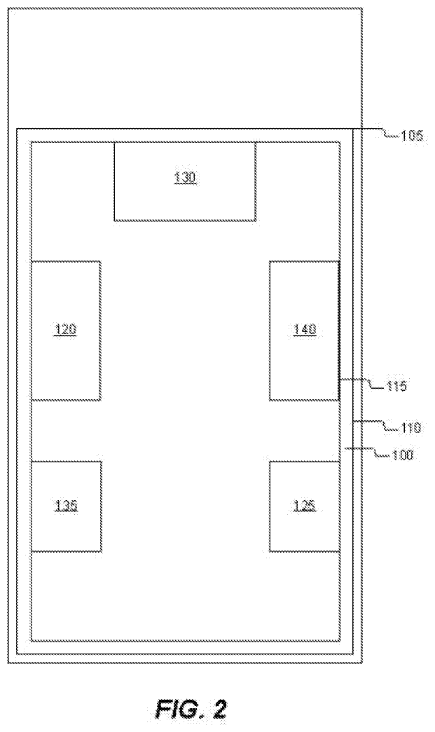

Referring now to FIGS. 1-2, shown therein is a mobile physical therapy apparatus (100). The mobile physical therapy apparatus (100) to be transported by a mobile physical therapy vehicle (105). The mobile physical therapy apparatus (100) including: a housing (110) detachably connected to an interior (115) of the mobile physical therapy vehicle (105); a treadmill (120) positioned within and detachably connected to the housing (110) of the mobile physical therapy vehicle (105); a recumbent bicycle (125) positioned within and detachably connected to the housing (110) of the mobile physical therapy vehicle (105); a pulley tower weight training system (130) positioned within and detachably connected to the housing (110) of the mobile physical therapy vehicle (105); an electrical stimulation and ultrasound apparatus (135) positioned within and detachably connected to the housing (110) of the mobile physical therapy vehicle (105); and a movable patient support table (140) positioned within and detachably connected to the housing (110) of the mobile physical therapy vehicle (105)

The mobile physical therapy apparatus (100) may be constructed from any material sufficient to function as described and claimed herein. For example, the mobile physical therapy apparatus (100) may be constructed from metals, plastics, wood, non-metals, alloys, resins, composite materials, organic compounds, non-organic compounds, papers, fabrics, combinations thereof and the like. The mobile physical therapy apparatus (100) may be constructed from a unitary piece of material or may be constructed from a variety of different materials. The mobile physical therapy apparatus (100) may also include reinforcing or bracing structures, such as ribs, struts, or braces for example or combinations thereof.

Further, the shape of the mobile physical therapy apparatus (100) may be any shape, including, but not limited to, a rectangular, square or circular shape for example. As shown in FIG. 2, the shape of the mobile physical therapy apparatus (100) is generally rectangular, so as to fit the general shape of the mobile physical therapy vehicle (105). In this way, the mobile physical therapy apparatus (100) may be selectively connected and disconnected and selectively removed from the mobile physical therapy vehicle (100). Thus, permitting the mobile physical therapy apparatus (100) to be utilized with a variety of different sized and shaped vehicles, including but not limited to the mobile physical therapy vehicle (105), for example. The mobile physical therapy apparatus (100) may be detachably connected to the interior of the mobile physical therapy vehicle (105) by any means known in the art, including but not limited to, bolts, screws, nails, joints, snaps, pins, adhesives and the like, for example.

The mobile physical therapy vehicle (105) is depicted in FIGS. 1 and 2 as a truck having a large enclosed interior portion capable of storing the mobile physical therapy apparatus (100). The mobile physical therapy vehicle (105) may be any vehicle capable of storing, securing and selectively removing, the mobile physical therapy apparatus (100). For example, trucks, vans, trailers, semi-trucks, cold-storage vehicles, and any other type of vehicle capable of functioning as described and claimed herein,

The housing (110) is an enclosure configured to support and store for use, a variety of different physical therapy equipment and is of a sufficient size and shape to permit patients and physical therapists to provide physical therapy within the housing (110) such that the physical therapy may be provided within the physical therapy vehicle (105). The housing (110) is detachably connected to an interior (115) of the mobile physical therapy vehicle (105), The housing (110) may be constructed from any material sufficient to function as described and claimed herein, For example, the housing (110) may be constructed from metals, plastics, wood, non-metals, alloys, resins, composite materials, organic compounds, non-organic compounds, papers, fabrics, combinations thereof and the like. The housing (110) may be constructed from a unitary piece of material or may be constructed from a variety of different materials. The housing (110) may also include reinforcing or bracing structures, such as ribs, struts, or braces for example or combinations thereof,

Further, the shape of the housing (110) may be any shape, including, but not limited to, a rectangular, square or circular shape for example. As shown in FIG. 2, the shape of the housing (110) is generally rectangular, so as to fit the general shape of the mobile physical therapy vehicle (105). In this way, the housing (110) may be selectively connected and disconnected and selectively removed from the mobile physical therapy vehicle (100). Thus, permitting the housing (110) to be utilized with a variety of different sized and shaped vehicles, including but not limited to the mobile physical therapy vehicle (105), for example. The housing (110) may be detachably connected to the interior of the mobile physical therapy vehicle (105) by any means known in the art, including but not limited to, bolts, screws, nails, joints, snaps, pins, adhesives and the like, for example.

The housing (110) may include heating and air conditioning, electrical components, lighting, running water, restrooms, and various other components necessary for the physical therapy apparatus to function as described and claimed herein. Components of the physical therapy apparatus (100) include but are not limited to a treadmill (120) positioned within and detachably connected to the housing (110) of the mobile physical therapy vehicle (105), a recumbent bicycle (125) positioned within and detachably connected to the housing (110) of the mobile physical therapy vehicle (105), a pulley tower weight training system (130) positioned within and detachably connected to the housing (110) of the mobile physical therapy vehicle (105), an electrical stimulation and ultrasound apparatus (135) positioned within and detachably connected to the housing (110) of the mobile physical therapy vehicle (105), and a movable patient support table (140) positioned within and detachably connected to the housing (110) of the mobile physical therapy vehicle (105). In some embodiments, the physical therapy equipment described herein is configured to be selectively removable and selectively detachable such that each piece of equipment may be selectively added, removed and rearranged within the housing (110) of the mobile physical therapy vehicle (105) depending upon the needs of the patient. Further, in some embodiments, the physical therapy equipment is configured to be selectively movable or slidable within the housing (110) of the mobile physical therapy vehicle (105), from a stowed position (when not in use), to a deployed position for use by a patient. In this way, the mobile physical therapy equipment may be customizable, based on patient needs, for any type of physical therapy.

Further, in some embodiments, the physical therapy equipment described herein, including specifically, the pulley tower weight training system (130) is configured to be locked or fixed in place and secured to the housing (110) of the mobile physical therapy vehicle (105) for added stability and safety during use. The physical therapy equipment described herein. including the pulley tower weight training system (130) may be locked in place using bolts, screws, pins, welded joints, straps and locks, combinations thereof and the like for example.

In some embodiments, the mobile physical therapy apparatus (100) may also include a control pad (116) configured to operate the various components of the mobile physical therapy apparatus (100) and mobile physical therapy vehicle (105). The control pad (116) may be operated manually inside the mobile physical therapy vehicle (105) using touch controls, automatically using a variety of pre-set functions, or remotely at a different location, via a computer operating through a wireless Internet connection, over the world wide web, or over a private network, radio waves, Bluetooth® connection, or the like, for example. The control pad (116) is a computer or similar electronic device configured for storing, sending, receiving and processing data according to instructions given to it by a computer program. In some embodiments, the control pad (116) may include a touch screen that displays patient data and other information on demand and in real-time. Thus, permitting a user of the mobile physical therapy apparatus to provide enhanced physical therapy by accessing and utilizing date real time during the physical therapy.

Likewise, each component detachably connected to the mobile physical therapy apparatus (100) is configured to store real time data associated with its use by a patient, upload such data directly to a computer and associate such data with a particular patient file. Including, for example, the treadmill (120), recumbent bicycle (125), pulley tower weight training system (130), electrical stimulation and ultrasound apparatus (135) and movable patient support table (140). In this way, when a patient utilizes the physical therapy equipment, the results may be automatically recorded, stored, displayed, and may be output into a report or similar format, in electronic or a paper form, for review by the physical therapist and the patient and automatically stored with the patients electronic medical records. Thus, greatly enhancing the feedback associated with the physical therapy and permitting a patient to see real time results based upon and in connection with their personal medical history and consistent with their physical therapy plan and other medical directives and recommendations, Thereby increasing the results of the physical therapy and improving the overall health of the patient.

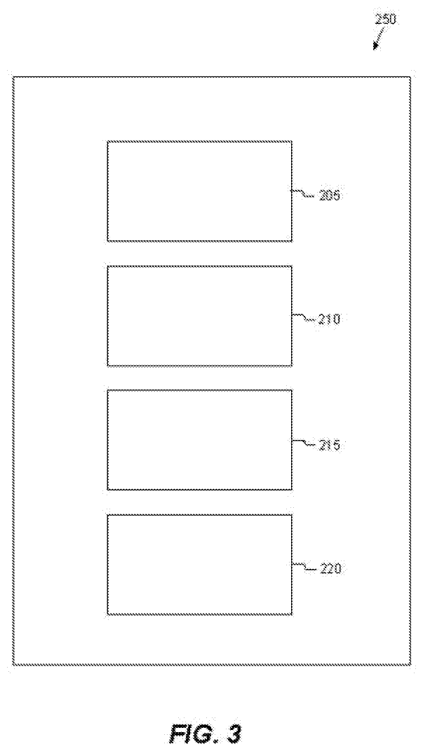

Referring now to FIG. 3, shown therein is a diagram depicting a method (200) for delivering mobile physical therapy. The method (200) includes the steps of: receiving (205) a prescription to provide physical therapy to a patient; confirming (210) that the prescription was prescribed by an authorized health care professional; scheduling (215) an appointment with the patient to provide mobile physical therapy to the patient; and providing (220) physical therapy to the patient in a mobile physical therapy vehicle (105).

In operation, a patient obtains a prescription for physical therapy services from a licensed healthcare provider. The prescription is then provided to a licensed physical therapist and confirmed as valid. The licensed physical therapist then schedules an appointment with the patient for the mobile physical therapy. The physical therapy is then provided by a licensed physical therapist at a date, time and location selected by the licensed physical therapist and the patient utilizing the mobile physical therapy apparatus (100) and mobile physical therapy vehicle (105).

A licensed physical therapist may provide, for example, a physical examination and evaluation of the patient, including a review of health history and certain testing procedures, including evaluating posture, movement, flexibility and muscle and joint motion and performance. A physical therapist may also provide a diagnosis, plan of care and short and long-term goals for improving a patient's physical abilities. The physical therapy prescribed may include specially designed exercises and use of specially designed equipment detachably connected to the mobile physical therapy apparatus (100) and mobile physical therapy vehicle (105).

For example, a patient may be prescribed use of the treadmill (120) positioned within and detachably connected to the housing (110) of the mobile physical therapy vehicle (105), recumbent bicycle (125) positioned within and detachably connected to the housing (110) of the mobile physical therapy vehicle (105), pulley tower weight training system (130) positioned within and detachably connected to the housing (110) of the mobile physical therapy vehicle (105), electrical stimulation and ultrasound apparatus (135) positioned within and detachably connected to the housing (110) of the mobile physical therapy vehicle (105), or the movable patient support table (140) positioned within and detachably connected to the housing (110) of the mobile physical therapy vehicle (105).

In some embodiments, the licensed physical therapist may view patient medical records, physical therapy history and results on the display screen of the control pad (116). The control pad (116) configured to operate the various components of the mobile physical therapy apparatus (100) and mobile physical therapy vehicle (105). The control pad (116) may be operated manually inside the mobile physical therapy vehicle (105) using touch controls, automatically using a variety of pre-set functions, or remotely at a different location, via a computer operating through a wireless internet connection, over the world wide web, or over a private network, radio waves, Bluetooth® connection, or the like, for example. The touch screen of the control pad (116) may be used to display patient data and other information on demand and in real-time. Thus, permitting a licensed physical therapist and patient to view and access real time data associated with the physical therapy provided in the mobile physical therapy apparatus (100) and mobile physical therapy vehicle (105).

As described above, each component detachably connected to the mobile physical therapy apparatus (100) is configured to store real time data associated with its use by a patient, upload such data directly to a computer and associate such data with a particular patient file. Including, for example, the treadmill (120), recumbent bicycle (125), pulley tower weight training system (130), electrical stimulation and ultrasound apparatus (135) and movable patient support table (140). In this way, when a patient utilizes the physical therapy equipment, the results may be automatically recorded, stored, displayed, and may be output into a report or similar format, in electronic or a paper form, for review by the physical therapist and the patient and automatically stored with the patients electronic medical records. Thus, greatly enhancing the feedback associated with the physical therapy and permitting a patient to see real time results based upon and in connection with their personal medical history and consistent with their physical therapy plan and other medical directives and recommendations. Thereby increasing the results of the physical therapy and improving the overall health of the patient. Further, in some embodiments, the physical therapy equipment described herein is configured to be selectively removable and selectively detachable such that each piece of equipment may be selectively added, removed and rearranged within the housing (110) of the mobile physical therapy vehicle (105) depending upon the needs of the patient. Further, in some embodiments, the physical therapy equipment is configured to be selectively movable or slidable within the housing (110) of the mobile physical therapy vehicle (105), from a stowed position (when not in use), to a deployed position for use by a patient. In this way, the mobile physical therapy equipment may be customizable, based on patient needs, for any type of physical therapy.

In some embodiments, the physical therapy equipment described herein, including specifically, the pulley tower weight training system (130) is configured to be locked or fixed in place and secured to the housing (110) of the mobile physical therapy vehicle (105) for added stability and safety during use. The physical therapy equipment described herein, including the pulley tower weight training system (130) may be locked in place using bolts, screws, pins, welded joints, straps and locks. combinations thereof and the like for example.

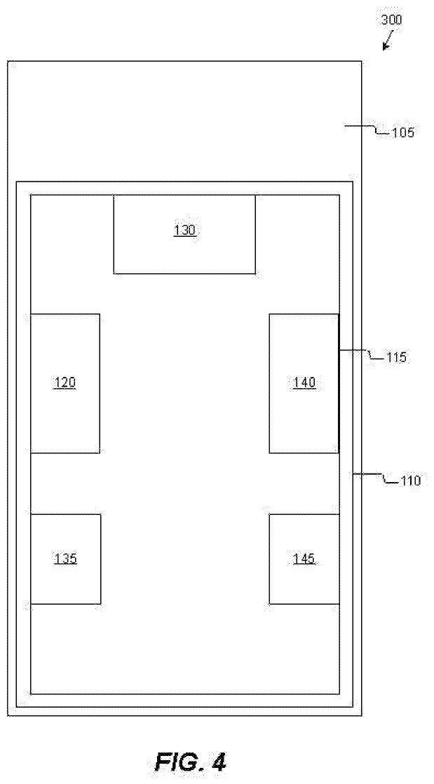

Referring now to FIG. 4, shown therein is a system (300) for providing mobile physical therapy. The system (300) includes a mobile physical therapy vehicle (105). The mobile physical therapy vehicle including a treadmill (120) positioned within and detachably connected to an interior portion of the mobile physical therapy vehicle (105); a recumbent bicycle (125) positioned within and detachably connected to an interior portion of the mobile physical therapy vehicle (105); a pulley tower weight training system (130) positioned within and detachably connected to an interior portion of the mobile physical therapy vehicle (105); an electrical stimulation and ultrasound apparatus (135) positioned within and detachably connected to an interior portion of the mobile physical therapy vehicle (105); and a movable patient support table (140) positioned within and detachably connected to an interior portion of the mobile physical therapy vehicle (105).

As described above with regards to FIGS. 1-2, the mobile physical therapy vehicle (105) is depicted as a truck having a large enclosed interior portion capable of storing the mobile physical therapy apparatus (100). The mobile physical therapy vehicle (105) may be any vehicle capable of storing, securing and selectively removing, the mobile physical therapy apparatus (100). For example, trucks, vans, trailers, semi-trucks, cold-storage vehicles, and any other type of vehicle capable of functioning as described and claimed herein.

The mobile physical therapy vehicle (105) may include heating and air conditioning, electrical components, lighting, running water, restrooms, and various other components necessary for the physical therapy apparatus to function as described and claimed herein. The mobile physical therapy vehicle (105) may include a treadmill (120) positioned within and detachably connected to the housing (110) of the mobile physical therapy vehicle (105), a recumbent bicycle (125) positioned within and detachably connected to the housing (110) of the mobile physical therapy vehicle (105), a pulley tower weight training system (130) positioned within and detachably connected to the housing (110) of the mobile physical therapy vehicle (105), an electrical stimulation and ultrasound apparatus (135) positioned within and detachably connected to the housing (110) of the mobile physical therapy vehicle (105), and a movable patient support table (140) positioned within and detachably connected to the housing (110) of the mobile physical therapy vehicle (105). In some embodiments, the physical therapy equipment described herein is configured to be selectively removable and selectively detachable such that each piece of equipment may be selectively added, removed and rearranged within the mobile physical therapy vehicle (105) depending upon the needs of the patient. Further, in some embodiments, the physical therapy equipment is configured to be selectively movable or slidable within the mobile physical therapy vehicle (105), from a stowed position (when not in use), to a deployed position for use by a patient. In this way, the mobile physical therapy equipment may be customizable, based on patient needs, for any type of physical therapy.

In some embodiments, the physical therapy equipment described herein, including specifically, the pulley tower weight training system (130) is configured to be locked in place and secured to the mobile physical therapy vehicle (105) for added stability and safety during use. The physical therapy equipment described herein, including the pulley tower weight training system (130) may be locked in place using bolts, screws, pins, welded joints, straps and locks, combinations thereof and the like for example.

In some embodiments, the mobile physical therapy vehicle (105) may also include a control pad (116) configured to operate the various components of the mobile physical therapy vehicle (105). The control pad (116) may be operated manually inside the mobile physical therapy vehicle (105) using touch controls, automatically using a variety of pre-set functions, or remotely at a different location, via a computer operating through a wireless internet connection, over the world wide web, or over a private network, radio waves, Bluetooth® connection, or the like, for example. The control pad (116) is a computer or similar electronic device configured for storing, sending, receiving and processing data according to instructions given to it by a computer program. In some embodiments, the control pad (116) may include a touch screen that displays patient data and other information on demand and in real-time. Thus, permitting a user of the mobile physical therapy apparatus to provide enhanced physical therapy by accessing and utilizing date real time during the physical therapy.

Likewise, each component detachably connected to the mobile physical therapy vehicle (105) is configured to store real time data associated with its use by a patient, upload such data directly to a computer and associate such data with a particular patient file. Including, for example, the treadmill (120), recumbent bicycle (125), pulley tower weight training system (130), electrical stimulation and ultrasound apparatus (135) and movable patient support table (140). In this way, when a patient utilizes the physical therapy equipment, the results may be automatically recorded, stored, displayed, and may be output into a report or similar format, in electronic or a paper form, for review by the physical therapist and the patient and automatically stored with the patients electronic medical records. Thus, greatly enhancing the feedback associated with the physical therapy and permitting a patient to see real time results based upon and in connection with their personal medical history and consistent with their physical therapy plan and other medical directives and recommendations. Thereby increasing the results of the physical therapy and improving the overall health of the patient.

It is to be appreciated that embodiments of the inventive concepts disclosed herein may be shipped fully or partially assembled, or fully or partially disassembled in the form of a kit, as will be readily appreciated by persons of ordinary skill in the art having the benefit of the instant disclosure.

From the above description, it is clear that the inventive concepts disclosed herein are adapted to carry out the objects and to attain the advantages mentioned herein as well as those inherent in the inventive concepts disclosed herein. While exemplary embodiments of the inventive concepts disclosed herein have been described for purposes of this disclosure, it will be understood that numerous changes may be made which will readily suggest themselves to those skilled in the art and which are accomplished within the broad scope of the inventive concepts disclosed herein and defined by the appended claims.

What is claimed is:

1. A mobile physical therapy apparatus to be transported by a mobile physical therapy vehicle, the mobile physical therapy apparatus comprising:
a housing detachably connected to an interior of the mobile physical therapy vehicle;
a treadmill positioned within and detachably connected to the housing of the mobile physical therapy vehicle;
a recumbent bicycle positioned within and detachably connected to the housing of the mobile physical therapy vehicle;
a pulley tower weight training system positioned within and detachably connected to the housing of the mobile physical therapy vehicle;
an electrical stimulation and ultrasound apparatus positioned within and detachably connected to the housing of the mobile physical therapy vehicle; and
a movable patient support table positioned within and detachably connected to the housing of the mobile physical therapy vehicle.

2. The mobile physical therapy apparatus of claim 1, further comprising a control pad configured to automatically operate the mobile physical therapy apparatus and display physical therapy data in real time.

3. A method for delivering mobile physical therapy, the method comprising the steps of:
receiving a prescription to provide physical therapy to a patient;
confirming that the prescription was prescribed by an authorized health care professional;
scheduling an appointment with the patient to provide mobile physical therapy to the patient; and
providing physical therapy to the patient in a mobile physical therapy vehicle.

4. A system for providing mobile physical therapy, the system comprising:
a mobile physical therapy vehicle comprising:
a treadmill positioned within and detachably connected to an interior portion of the mobile physical therapy vehicle;
a recumbent bicycle positioned within and detachably connected to an interior portion of the mobile physical therapy vehicle;
a pulley tower weight training system positioned within and detachably connected to an interior portion of the mobile physical therapy vehicle;
an electrical stimulation and ultrasound apparatus positioned within and detachably connected to an interior portion of the mobile physical therapy vehicle; and
a movable patient support table positioned within and detachably connected to an interior portion of the mobile physical therapy vehicle.

5. The system for providing mobile physical therapy of claim 4 further comprising a control pad configured to automatically operate the mobile physical therapy apparatus and display physical therapy data in real time.
